(19) United States
(12) Patent Application Publication
(10) Pub. No.: US 2018/0344145 A1
(43) Pub. Date: Dec. 6, 2018

(54) METHOD AND APPARATUS FOR COLPOSCOPIC EXAMINATION

(71) Applicants: Danylo KOZUB, Kharkiv (UA); Iurii SHAPOVAL, Kharkiv (UA); Sergii PUSTOVAROV, Kyiv (UA)

(72) Inventors: Danylo KOZUB, Kharkiv (UA); Iurii SHAPOVAL, Kharkiv (UA); Sergii PUSTOVAROV, Kyiv (UA)

(21) Appl. No.: 15/611,831

(22) Filed: Jun. 2, 2017

(51) Int. Cl. A61B 1/307; G02B 7/09; G02B 7/28; A61B 1/00; A61B 1/04

(52) U.S. Cl. CPC A61B 1/307; G02B 7/09; G02B 7/28; A61B 1/00016; A61B 1/04; A61B 1/00193; A61B 1/00009; A61B 1/00188

(57) ABSTRACT

The colposcopic examination apparatus comprising an optical head with a lens capable of changing focusing distance and an image acquiring unit capable of registering images created by the lens, as well as a computer system designed to control the lens and the image acquiring unit, to obtain a series of images created by the lens at different focusing distances and to perform focus stacking of the obtained images, wherein the computer system is capable of automatic recognition of the chosen examined object on the image created by the lens and of further automatic setting of shooting parameters, focusing distance of the lens and number of images to be created by the lens at different focus distances for the computer system to perform focus stacking and to generate the output image of the examined object with extended depth of field. Also disclosed is a method of colposcopic examination.

### FIELD OF THE INVENTION

The invention relates to medicine, specifically to gynecology, in particular to the method and the apparatus for colposcopic examination involving processing of digital images.

### BACKGROUND OF THE INVENTION

Colposcopic examination is based on revealing the difference in mucosal relief and vessel appearance in normal and pathological epithelium of the vaginal portion of cervix and is an efficient method to detect lesions of ectocervical and endocervical epithelium, to assess their nature and localization, to perform differential diagnosis of benign lesions of ectocervix, vagina and vulva, to assess feasibility of cervical biopsy, to determine the area and the method of histological examination sampling, to choose the treatment method of the identified pathology.

Obtaining colposcopic images presents difficulties that have not been solved by the prior art. Normally the apparatus for colposcopic examination is positioned at a small distance from the chosen object (15-30 cm). Such range allows to obtain high optical resolution, sufficient illumination of the object, at the same time ensuring access for the doctor's manipulations, such as applying solutions, tissue sampling etc. At such a range the depth of field, which is the zone where examined objects appear acceptably sharp, constitutes from several millimeters for small matrix cameras (⅓ inch) to tenths of a millimeter for full frame cameras. Quality and resolution are normally proportional to the matrix size, i.e. the depth of field decreases as the image quality requirements grow.

At the same time, shallow depth of field is a negative factor when using images obtained during of colposcopic examination, since just a small part of the examined object is acceptably sharp on each of the images. Several frames to several dozens of frames are needed to obtain information about the entire object. Large number of frames complicates their further analysis and increases probability of some of the examined object areas being captured in quality too low for further analysis.

One of the prior art solutions of the shallow depth of field issue is the focus stacking method, a tool allowing to extend the depth of field of a digital image. This method is based on merging multiple source images of the same scene, usually captured from the same point of view with different depth of field (for instance, a series of static images captured from a fixed point of view or a series of frames from a stationary video camera), to produce an output image with a depth of field larger than on any of the source images.

In order to render the entire examined object sharp through focus stacking, source images should meet certain requirements. In particular, the areas of depth of field of subsequent source images should have overlapping areas in order to provide sufficient data. Besides, some focus stacking methods require that the depth of field area of the series of output images does not change the shift direction (i.e. the focus has to be shifted in the same direction—either towards infinity or camera).

Capturing images for focus stacking is called focus bracketing. Normally each frame in focus bracketing is captured with the same settings as the previous one, but the focus point of the lens, and therefore, the depth of field, is being shifted by a specified step in a specified direction. Most often focus bracketing is done according to the following procedure: the first frame is taken with focus at the nearest point of the object, the following ones are captured with the shift of focus towards infinity until the farthest point of the object is in focus.

Performing manual focus bracketing of images captured during colposcopic examination is problematic. The major problem is to determine the nearest and the farthest points of the examined object. Mucosa surfaces are normally characterized by low contrast level and it is quite difficult for the specialist conducting examination to define if the relevant area is in focus or not. In addition, even examination of such a simple three-dimensional figure as a raised hemisphere of the cervix surface present difficulties for determining the nearest point of the examined object, due to the cervix often being inclined, deformed and non-uniformly illuminated. Colposcopic examination of other objects involves even more difficulties due to their three-dimensional shape.

### SUMMARY OF THE INVENTION

According to the present invention, the problem of the prior art is solved by developing a method and an apparatus for colposcopic examination.

In the first aspect the claimed invention is an apparatus comprising optical head with a lens designed with possibility to modify focusing distance, and an image acquiring unit capable of registering images created by the lens, as well as a computer system designed to control the lens and the image acquiring unit, to obtain a series of images created by the lens at different focusing distances and to perform focus stacking of the obtained images, wherein the computer system is capable of automatic recognition of the chosen examined object on the image created by the lens and of further automatic setting of shooting parameters, focusing distance of the lens and number of images to be created by the lens at different focus distances for the computer system to perform focus stacking and to generate the output image of the examined object with extended depth of field.

In the second aspect, the claimed invention is the method of conducting colposcopic examination comprising selection of the examined object, obtaining series of images created by the lens at different focusing distances by a computer system of the apparatus for colposcopic examination, and performing focus stacking of the created images. Once the examined object has been selected it's automatically recognized on the image created by the lens, with further automatic setting of shooting parameters, focusing distance of the lens and number of images created by the lens at different focusing distances required for the computer system to perform focus stacking and to generate the output image of the examined object with extended depth of field.

The Summary of the Invention is provided to introduce the main concept of the invention in a simplified form that is further described below in the Detailed Description of the Invention. The Summary of the Invention is not intended to identify essential features of the claimed subject matter, nor is it intended to be used to limit the scope of the claimed subject matter.

### BRIEF DESCRIPTION OF THE DRAWINGS

FIG. 1 is a block diagram of an example embodiment of the method of colposcopic examination according to one of the preferred embodiments of the present invention.

FIG. 2 is an example of a source image created by the lens with certain focusing distance in its first position, where a part of gynecological speculum is in the area of depth of field.

FIG. 3 is an example of a source image created by the lens at different focusing distances in its first position, where a portion of cervix is in the area of depth of field.

FIG. 4 is an example of a source image created by the lens with certain focusing distance in its second position, where a part of gynecological speculum is in the area of depth of field.

FIG. 5 is an example of a source image created by the lens with certain focusing distance in its second position, where the portion of cervix closest to the lens is in the area of depth of field.

### DETAILED DESCRIPTION OF THE INVENTION

There are different apparatus for colposcopic examination producing colposcopic images of the examined object that require further digital processing.

Pursuant to the present invention, the apparatus comprises an optical head, a lens with possibility of changing focusing distance, and an image acquiring unit capable of registering images created by the lens, as well as a computer system capable of controlling the lens and the image acquiring unit, acquiring series of images created by the lens at different focusing distances and performing focus stacking of produced images, wherein the computer system is capable to automatically recognize the preselected examined object on the image created by the lens, with further automatic setting of shooting parameters, focusing distance of the lens and number of images to be created by the lens at different focusing distances, required for the computer system to perform focus stacking and to produce the output image of the examined object with extended depth of field.

According to the preferred embodiment of the invention, the computer system comprises at least one processor and at least one computer readable medium communicatively connected with at least one processor, as well as program instructions stored on at least one computer readable medium and executable by at least one processor.

The program instructions described herein are executable by at least one or several hardware logic components. Examples of possible types of hardware logic components that can be used (without limitation) include field-programmable gate arrays (FPGAs), application-specific integrated circuits (ASICs), systems-on-a-chip (SOCs), complex programmable logic devices (CPLDs), etc. Program instructions for carrying out operations of the present invention may be represented by assembler code instructions, instruction-set-architecture (ISA) instructions, machine instructions, machine-dependent instructions, set of micro commands, base software instructions, state data, or another source code or object code written in any combination of one or more programming languages, including object oriented programming languages such as Smalltalk, C++ or the like, and conventional procedural programming languages such as C or C-like programming languages.

The program instructions may be executed entirely on the user's computer being a part of the computer system, partly on the user's computer being a part of the computer system as a stand-alone software, partly on the user's computer being a part of the computer system and partly on a remote computer or entirely on a remote computer or server. In the latter scenario, the remote computer may be connected to the user's computer through any type of network, including local area network (LAN) and wide area network (WAN), or it can be connected to an external computer (for example, through the Internet using an Internet Service Provider).

In some embodiments, for example, in electronic circuitry, programmable logic devices, field-programmable gate arrays (FPGA), or programmable logic arrays (PLA) can execute computer readable program instructions optimizing the information about the state of such instructions for adjustment of electronic circuitry in order to implement the aspects of the present invention.

The computer readable medium includes volatile and non-volatile, removable and non-removable media applicable in any method or technology of storing such information as computer readable instructions, data structures, program modules, etc.

Computer readable medium may include random access memory (RAM), read-only memory (ROM), electrically-erasable programmable ROM (EEPROM), flash memory or another memory technology, CD-ROM, DVD format compact disc or another optical storage, magnetic cassettes, magnetic tape, magnetic disk storage or other magnetic storage device, as well as any other non-transmission medium that can be used to store information for access by a computing apparatus. Said computer readable storage medium is a non-transitory one.

According to the preferred embodiment of the present invention, the computer system is capable of automatic setting of shooting parameters chosen from a group that in the preferred embodiment of this invention includes focusing distance value, aperture, sensitivity, exposure time values, white balance settings, brightness level of flash or additional illumination, though this settings list is not exhaustive.

According to the preferred embodiment of the present invention, the computer system has possibility to automatically recognize the examined object through at least two iterations, where the lens creates images for preliminary recognition of the examined object and further refinement of obtained data about the examined object. The computer system automatically sets shooting parameters for at least one iteration during automatic recognition of the examined object.

Also according to the preferred embodiment of the present invention, the computer system is capable of using data selected from the group including examined object data, data on additional gynecological equipment parameters, patient data, as well as capable of acquisition and storage of the patient's colposcopic examination results data.

Pursuant to another preferred embodiment of the present invention, the computer system can generate a three-dimensional model of the examined object based on the obtained images.

Also according to the preferred embodiment of the present invention, the computer system can position the optical head relative to the selected examined object to ensure optimal shooting conditions for the examined object.

Pursuant to yet another preferred embodiment of the present invention, the computer system can combine the current output image of the examined object with extended depth of field with previously obtained output image of the examined object with extended depth of field to detect changes in the examined object over time.

According to the preferred embodiment of the present invention, the claimed apparatus is designed to conduct colposcopic examination of objects selected from a group that includes areas adjacent to vaginal opening, vaginal opening, vagina walls, ectocervix and endocervix for further detection and classification of skin and mucosa lesions.

The preferred method of automatic recognition of the examined object is as follows. In cases when the examined object requires using the gynecological speculum as additional gynecological equipment mentioned above, preliminary recognition is performed through matching of sample images that may be used by the computer system with the current source image. So the data about the type and the size of currently used gynecological speculum is obtained through interaction of the user, for instance, with the user interface of the computer system of the colposcopic examination apparatus used for implementation of the claimed method, and selection by the user of the required parameters from the list of available parameters characterizing additional gynecological equipment. The user should have possibility to choose the type and the size of currently used gynecological speculum in the settings. With all the available information about the parameters of gynecological speculum the computer system of the claimed apparatus with higher accuracy will perform automatic recognition of the gynecological speculum on the image created by the lens and will determine the exact distance to the gynecological speculum, the angle to the gynecological speculum axis and the approximate distance to the cervix being the examined object. All this information can be used for further setting of optimal shooting parameters, lens focusing distance, number of images etc. In particular, the computer system with the positioning unit ensures positioning of the optical head by generating a command for its movement (lens movement) so that the distance and the direction are optimal for the current examined object. Besides, the lens focusing distance shift step, the aperture value, the sensitivity depend on illumination level. For instance, the vaginal depth, the material and design of gynecological speculum significantly affect illumination level when photographing cervix. At high illumination level, it's optimal to use small aperture values (f11-f13) and larger lens focusing distance shift step value (focus bracketing step). At low illumination level, it's better to use high aperture value (f5.6) and smaller lens focusing distance shift step. After preliminary recognition of the gynecological speculum and determining its position, one or several additional iterations may be required to define the position of the examined object more precisely. For instance, after correct positioning of the lens through movement of the optical head and setting of new shooting parameters, it's needed to precisely determine the nearest and farthest point of the examined object. For cervix, this task is more complicated due to the mucosal surfaces having very little detail. In this case the apparatus may rely on recognition of cervical canal and the preset information about the physiological cervix shape instead of recognition of contrast details.

Unlike conventional pattern recognition such as detecting faces on the image, implementation of the present invention may require performing at least two iterations for preliminary recognition of the object using all the available information, for instance, gynecological speculum parameters, physiological shape of cervix and other parts. This is especially vital if the examined object does not require using additional gynecological equipment. The information used may also include patient data (height, weight, race, age), as well as data on the patient's previous colposcopic examinations collected and stored by the computer system of the apparatus. Preferably, matching of sample images that can be used by the computer system with the current image created by the lens should be performed during preliminary recognition.

The three-dimensional model of the examined object obtained after automatic recognition and the information about the position of the apparatus optical head and the lens itself relative to the examined object allows to ensure further positioning of the optical head relative to the chosen examined object in order to provide optimal shooting conditions for the examined object. Positioning can be ensured through installation of the positioning unit on the colposcopic examination apparatus allowing for automatic change of the optical head position after the computer system receives the corresponding command from the user entered through computer interface, or by providing the required information to the user through user interface of the computer system with further manual positioning of the optical head.

The present invention is also described in the present patent claim through the colposcopic examination method implemented with the help of the above described apparatus.

The aspects of the present invention are described herein with reference to the drawings.

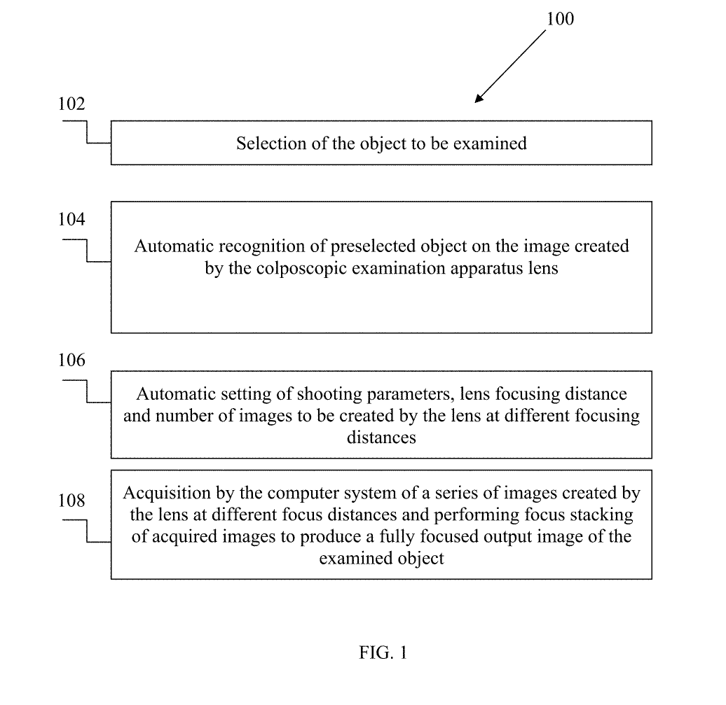

FIG. 1 depicts a block diagram of an example implementation of the colposcopic examination method 100 according to one of the preferred embodiments of the present invention. Said method 100 comprises examples of specific steps of colposcopic examination. Some steps of the method 100 or the entire method 100 (or any other processes described herein, or variations and/or combinations thereof) can be implemented as program instructions (for instance, program code, one or several computer programs or one or several applications) executed collectively by one or several processors, computer system hardware or the combination thereof. Program instructions can be stored on one or more computer readable media, for instance, as a computer program containing multiple instructions executed by one or more processors. Computer readable information storage medium is a non-transitory one.

Block diagram of the method 100 begins with the block 102 illustrating the step of selecting the object to be examined. The examined object can be selected by the computer system of the colposcopic examination apparatus receiving data about the object chosen by the user from the list of available objects, for instance, through interaction of the user, for instance, with the user interface of the computer system of the colposcopic examination apparatus implementing the said method 100.

The next block diagram 104 depicts automatic recognition of the preliminary selected examined object on the image created by the colposcopic examination apparatus lens. As described above, the preferred method of automatic recognition of the examined object may consist in the following. In cases when the examined object requires using gynecological speculum as above mentioned additional gynecological equipment, the preliminary recognition is performed by matching sample images that can be used by the computer system, with the current source image created by the lens, wherein the above mentioned sample images can be uploaded to the computer system by the manufacturer or user and be stored there. Data about the type and the size of currently used gynecological speculum is obtained through interaction of the user with, for instance, the user interface of the computer system of the colposcopic examination apparatus implementing the claimed method 100 and user selection of required parameters from the list of parameters characterizing available additional gynecological equipment. The gynecological speculum is automatically recognized on the image created by the lens using available information about the parameters of gynecological speculum, and the precise distance to the gynecological speculum, the angle relative to the gynecological speculum axis and the approximate distance to cervix being, for instance, the examined object, are determined. Automatic recognition of the examined object can also be performed without using additional gynecological equipment, in this case a larger number of iterations will be needed for preliminary recognition of the examined object and further refinement of the obtained data about the examined object.

The next is the block 106 depicting automatic setting of shooting parameters, focusing distance of the lens and the number of images created by the lens at different focusing distances. Once the operations of the block 104 have been performed, those of the block 106 should be performed on the basis of the obtained data for refinement of the examined object position or for creation of a series of images of the already recognized examined object for further digital processing, executing either the full range of described operations or setting just one of the above stated parameters depending on whether this operation is performed after full automatic recognition of the examined object or after one of iterations during automatic object recognition.

The next block 108 depicts the process of the colposcopic examination apparatus computer system acquiring a series of images created by the lens at different focusing distances and performing focus stacking of obtained images to produce the output image of the examined object with extended depth of field, wherein focus stacking can be performed in any of the known methods, for instance, by the method based on the depth map generation.

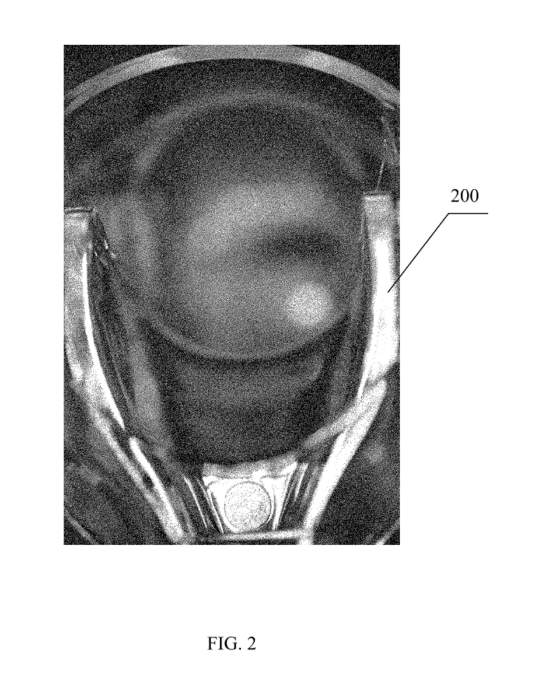

FIG. 2 depicts an image created by the lens in its first position when focusing at the examined object located 30 cm from the lens, with minimal aperture value, wherein the area of depth of field contains a part of gynecological speculum 200 with the parameters known to the user.

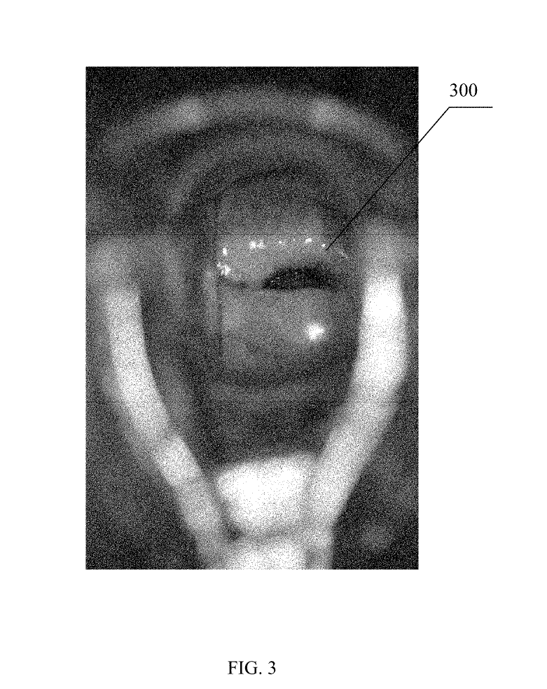

FIG. 3 depicts an image created by the lens in its first position, but with the focusing distance changed based on the data obtained by the computer system of the apparatus after processing of the image on the FIG. 2, wherein the portion of cervix 300, in particular the cervical canal, is in the area of depth of field.

Based on the analysis results of position of cervical canal and gynecological speculum from the FIG. 3 and FIG. 2 respectively, that is performed by the computer system of the claimed apparatus in course of automatic recognition of the examined object, a reasonably precise assumption about the position of the optical head and, respectively, the lens of the apparatus relative to the gynecological speculum axis is made. In order to photograph all portions of cervix, the lens should be positioned as close to the gynecological speculum axis as possible. Based on the information described above the position of the optical head and, respectively, the lens relative to the examined object is changed automatically, if allowed for by the apparatus design, or manually, and the above described operations are repeated in the new position.

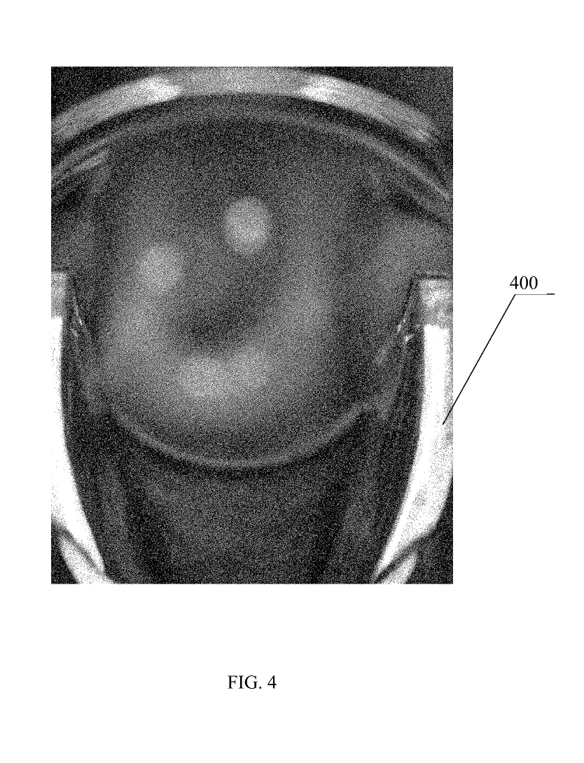

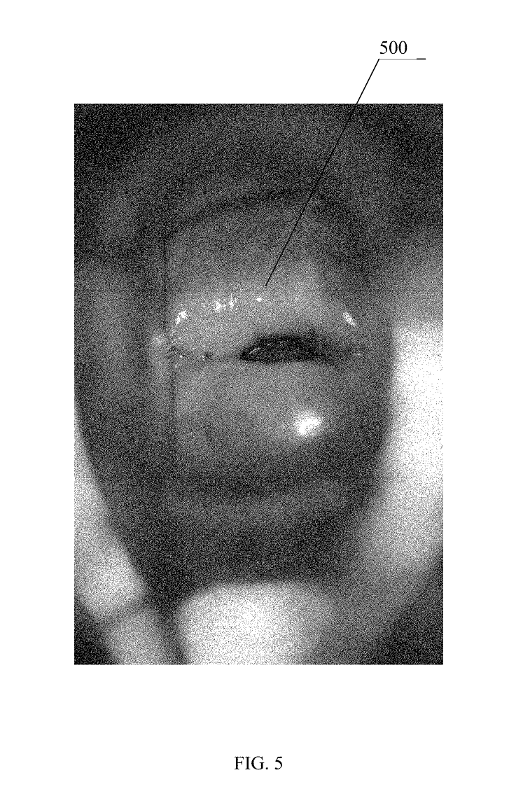

FIG. 4 depicts an image created by the lens with certain focusing distance in its second position with a part of gynecological speculum 400 in the area of depth of field, FIG. 5 depicts an image created by the lens with certain focusing distance in its second position with the portion of cervix 500 closest to the lens in the area of depth of field. These images demonstrate that this lens position is optimal, the cervix is not hidden by the edges of gynecological speculum and the portion of cervix closest to the apparatus is in focus. So further shooting of cervix is done in this position for the lens to shoot a series of images with different focusing distances required for the computer system to produce an image of the examined object with extended depth of field.

Although the examples of performing colposcopic examinations were described in a language specific to the structural features and/or methodological steps, it should be understood that the method of conducting colposcopic examination, as well as the colposcopic examination apparatus as defined in the appended claims are not necessarily limited to the specific features or steps described above. Rather, the specific features and steps described above are disclosed as examples of implementing the claims, and other equivalent features and steps can be encompassed by the claims of the present invention.

1. Apparatus for colposcopic examination, comprising
optical head comprising a lens capable of changing focusing distance and an image acquiring unit capable of registering images created by the lens,
as well as a computer system capable of controlling the lens and the image acquiring unit,
of producing series of images created by the lens at different focusing distances and performing focus stacking of produced images,
wherein
the computer system is capable of automatic recognition of the preliminary selected object on the image created by the lens,
and further automatic setting of shooting parameters, lens focusing distance and number of images to be created by the lens at different focusing distances, required to produce an output image with extended depth of field through performing of focus stacking by the computer system.

2. The apparatus of claim 1, characterized in that the computer system comprises at least one processor and at least one computer readable medium communicatively connected to at least one processor, as well as program instructions stored on at least one computer readable medium and executable by at least one processor.

3. The apparatus of claim 1, characterized in that the computer system is capable of automatic setting of shooting parameters selected from the group including focusing distance value, aperture, sensitivity, exposure time values, white balance settings, brightness level of flash or additional illumination.

4. The apparatus of claim 1, characterized in that the computer system is capable of automatic recognition of the examined object through performing of at least two iterations for preliminary recognition of the examined object and further refinement of obtained data about the examined object.

5. The apparatus of claim 4, characterized in that the computer system is capable of automatic setting of shooting parameters for at least one iteration during of automatic recognition of the examined object.

6. The apparatus of claim 1, characterized in that the computer system is capable of using data selected from the group including data about the examined object, about the parameters of additional gynecological equipment, the patient data.

7. The apparatus of claim 1, characterized in that the computer system is capable of collection and storage of data about the results of the patient's colposcopic examination.

8. The apparatus of claim 1, characterized in that the computer system is capable of creating a three-dimensional model of the examined object based on produced images.

9. The apparatus of claim 1, characterized in that the computer system is capable of positioning the optical head relative to the chosen examined object to ensure optimal shooting conditions of the examined object.

10. The apparatus of claim 1, characterized in that the computer system is capable of combining the current output image of the examined object with extended depth of field with a previously produced output image of the examined object with extended depth of field to detect changes of the examined object over time.

11. The apparatus of claim 1, characterized in that the apparatus is capable of performing colposcopic examination of objects selected from the group including areas adjacent to vaginal opening, vaginal opening, vagina walls, ectocervix and endocervix for further detection and classification of skin and mucosa lesions.

12. Method of performing colposcopic examination, comprising
selection of the object to be examined,
acquisition by the colposcopic examination apparatus computer system of a series of images created by the lens at different focusing distances, and performing focus stacking of acquired images,
wherein after selecting the examined object, the automatic recognition of preliminary selected examined object on the image created by the lens is performed,
as well as further automatic setting of shooting parameters, focusing distance of the lens and number of images to be created by the lens at different focusing distances, required to produce the output image of the examined object with extended depth of field through performing focus stacking by the computer system.

13. The method of claim 12, characterized in automatic setting of shooting parameters selected from the group including focusing distance value, aperture, sensitivity, exposure time values, white balance settings, brightness level of flash or additional illumination.

14. The method of claim 12, characterized in that the automatic recognition of the examined object is performed through at least two iterations for preliminary recognition of the examined object and further refinement of obtained data about the examined object.

15. The method of claim 14, characterized in that the automatic setting of shooting parameters is performed for at least one iteration during automatic recognition of the examined object.

16. The method of claim 12, characterized in that it comprises entry of the data selected from the group including data about the examined object, parameters of additional gynecological equipment, patient data.

17. The method of claim 12, characterized in performing collection and storage of the patient's colposcopic examination results data.

18. The method of claim 12, characterized in building of three-dimensional model of the examined object based on the obtained images.

19. The method of claim 12, characterized in positioning of the optical head of the colposcopic examination apparatus relative to the selected examined object in order to ensure optimal shooting conditions for the examined object.

20. The method of claim 12, characterized in combining the current output image of the examined object with extended depth of field with a previously produced output image of the examined object with extended depth of field to detect changes of the examined object over time.

21. The method of claim 12, characterized in conducting colposcopic examination of objects selected from a group that includes areas adjacent to vaginal opening, vaginal opening, vagina walls, ectocervix and endocervix for further detection and classification of skin and mucosa lesions.
